(19) United States
(12) Patent Application Publication
(10) Pub. No.: US 2003/0068639 A1
(43) Pub. Date: Apr. 10, 2003

(54) Detecting biochemical reactions

(76) Inventors: Thomas Haneder, Dachau (DE); Alfred Martin, Regensburg (DE); Volker Lehmann, Munich (DE); Thomas Ehben, Munich (DE); Hans Christian Hanke, Munich (DE); Karin Fuchs, Munich (DE)

Correspondence Address: FISH & RICHARDSON PC, 225 FRANKLIN ST, BOSTON, MA, 02110, US

(21) Appl. No.: 10/233,001

(22) Filed: Aug. 30, 2002

(30) Foreign Application Priority Data
Aug. 31, 2001 (DE) 10142691.7

(51) Int. Cl. C12Q 1/68; B05D 3/00; G01N 33/53; G01N 33/542; C12M 1/34

(52) U.S. Cl. 435/6; 435/7.9; 427/2.11; 435/287.2

(57) ABSTRACT

A method for detecting a biochemical reaction includes immobilizing a capture molecule on an inner wall of a pore selected from a multiplicity of pores extending between first and second opposed surfaces of a macroporous substrate and contacting an analyte with the capture molecule. A light is then directed into the pore. A change in a light transmission property of the pore is then detected. This change indicates a binding reaction between the analyte and the capture molecule.

### FIELD OF INVENTION

The present invention relates to a method for detecting biochemical reactions and to an apparatus therefor (“biochip” or “lab on chip”), in particular for studying DNA hybridization, protein-protein interactions and other binding reactions in the area of genome, proteome or active substance research in biology and medicine.

### RELATED APPLICATIONS

This application claims the benefit of the Aug. 31, 2001 priority date of German application 101 42 691.7-52.

### BACKGROUND

The detection of (bio)chemical reactions, i.e. the detection of biologically relevant molecules in defined test material, is of enormous importance to the biological sciences and medical diagnostics. In this context, “biochips” are undergoing continuous development. Such biochips are usually miniaturized hybrid functional elements with biological and technical components, in particular biomolecules immobilized on a surface (outer surface and/or inner surface), which serve as specific partners for interaction. The structure of these functional elements frequently has rows and columns. These are then called “chip arrays”. Since thousands of biological or biochemical functional elements may be arranged on a single chip, the latter are normally produced using microtechnical methods. Suitable biological and biochemical functional elements are in particular DNA, RNA, PNA (for example, single strands, triplex structures or combinations thereof may be present in nucleic acids and their chemical derivatives), saccharides, peptides, proteins (e.g. antibodies, antigens, receptors), derivatives of combinatorial chemistry (e.g. organic molecules), cell components (e.g. organelles), individual cells, multicellular organisms and cell assemblages.

At present, mainly optical methods are used in the area of biochips. In this connection, appropriate biological or biochemical reactions are detected, for example, by attaching small amounts of different capture molecules in the form of dots and matrices on a surface of, for example, glass or gold. An analyte to be studied, which can usually be fluorescently labeled, is then pumped across this surface. When the appropriate molecules of the fluorescently labeled analyte react with the capture molecules immobilized on the surface of the support substrate, this reaction can be detected by optical excitation using a laser and measurement of the corresponding fluorescence signal. However, a disadvantage of such an optical method is that the analyte must be labeled, i.e. must be provided with appropriate fluorescent molecules, for example Cy3, Cy5, or the like. On the one hand, this necessitates a chemical reaction between the analyte molecule and the fluorescent dye molecule. On the other hand, the emissivity of the fluorescent molecules is reduced during longer or repeated measurements, resulting in a reduction in the intensity of the measured signal. Furthermore, binding of the molecule used for labeling, e.g. fluorescent labeling, to the analyte can cause an undesirable change in the binding behavior of said analyte toward the capture molecules.

### SUMMARY

It is thus the object of the present invention to provide a simple, flexible and inexpensive method and an apparatus for detecting biochemical reactions by means of “lab on chips” or “biochips”, without having to label the analyte, i.e. the target molecules to be studied, which can therefore be used in native form.

In particular, a method for detecting biochemical reactions is provided, comprising the following steps:

(a) providing a macroporous substrate which has a first and a second surface opposite one another, with a multiplicity of discrete pores having a diameter in the range from 500 nm to 100 μm, preferably 5 to 10 μm, being arranged distributed over at least one surface area, which pores extend through the substrate from the first to the second surface,

(b) location-specific immobilizing or attaching per pore of at least one capture molecule on the inner wall surfaces of at least some of the pores, the capture molecule being capable of undergoing a biochemical reaction,

(c) contacting an analyte with the at least one capture molecule in at least one pore,

(d) illuminating the first surface with light, and

(e) measuring the light transmission property of the at least one pore, which changes as a function of the occurrence of a binding reaction between the analyte and the capture molecule immobilized on the inner wall surface of the at least one pore.

The present invention provides a novel technical platform for the inexpensive, flexible and reliable detection of biochemical reactions on the basis of “lab on chips” or “biochips”. The present invention, for the first time, makes possible an optical detection of biochemical reactions, without having to label the analyte to be studied, for example without the use of fluorescent molecules or other, for example radioactive, markers. Furthermore, there is the preferred possibility of high parallelization due to a large number of appropriate pores.

The present invention further relates to an apparatus for detecting biochemical reactions, comprising:

(a) at least one macroporous substrate, preferably macroporous silicon, which has a first and a second surface opposite one another, with a multiplicity of discrete pores having a diameter in the range from 500 nm to 100 μm, preferably 5 to 10 μm, being arranged distributed over at least one surface area, which pores extend through the substrate from the first to the second surface, with at least one capture molecule per pore, which molecule is capable of undergoing a biochemical reaction, being location-specifically immobilized on the inner wall surfaces of at least some of the pores,

(b) a light supply device for supplying light to the pores, and

(c) a measuring device for recording the light transmitted through the pores and for analyzing the light transmission property of the at least one pore, which changes as a function of the occurrence of a binding reaction between the analyte and the capture molecule immobilized on the inner wall surface of the at least one pore.

The arrangement pattern of the pores is designed, at least in some areas, according to a grid layout. The apparatus of the invention furthermore usually has automatic application and sampling devices which can be scanned in the X-Y direction and which are preferably microvalves which can be controlled from the outside and which are arranged in the same grid layout as the arrangement pattern of the pores. Furthermore, a support or bottom plate which has a recording device in the same arrangement for analysis on a microprocessor may be arranged below the second surface. Such a recording device may be a CCD array or another appropriate detection unit as is common in this specific field, which can also be arranged tilted at an angle α to the macroporous substrate or to the chip. Preferably, a CCD array is arranged below the second surface.

In one embodiment of the apparatus of the invention, the light supply device comprises at least one light waveguide which is arranged in such a way that light is coupled directly into at least one pore. In another embodiment of the apparatus of the invention, the light supply device comprises at least one light waveguide which is arranged in such a way that it covers a multiplicity of pores of the macroporous substrate. In this connection, both planar light waveguides and vertically emitting laser diodes may be provided. To couple out the light, the apparatus of the invention may comprise, for example, one or more glass fibers beveled by about 35° to 55°, preferably by about 45°. In another embodiment of the apparatus of the invention, the light supply device and the measuring device may be arranged on the side of the first surface and a reflecting agent which reflects light transmitted through the pores at least partially through the pores into the measuring device may be arranged on the side of the second surface.

### BRIEF DESCRIPTION OF THE DRAWINGS

The invention is described below on the basis of accompanying drawings of preferred embodiments, in which:

FIG. 1 shows an exemplary diagram of an arrangement for carrying out the method of the invention, in which a glass fiber 30 at defined distances leads in each case into a pore 11 or is closely positioned in each case above said pore 11;

FIG. 2 shows an arrangement in which a single glass fiber covers several pores of the macroporous substrate 10 used according to the invention, FIG. 2(A) being a bottom view of the substrate 10 and FIG. 2(B) being a sectional view through the arrangement;

FIG. 3(A) shows an arrangement in which a single glass fiber covers several pores 11 of the substrate 10; and

FIG. 3(B) shows an arrangement with regard to a planar light waveguide (32) which directs the transmitted signals to the side of the chip or substrate 10; and

FIG. 4 shows another arrangement in which the coupled-in light is detected only after reflection on the rear side or the second surface 10B of the substrate 10.

### DETAILED DESCRIPTION

The method of the invention and the apparatus of the invention may be used for detecting biochemical reactions in order to characterize or to identify in another way molecular species which are capable of binding in a controllable manner to biomolecules or capture molecules which have been immobilized on a macroporous substrate 10. This includes in particular antibody-antigen and ligand-receptor binding and the analysis of nucleic acid sequences. To this end, the macroporous substrate 10 has a multiplicity of pores or through holes or through channels or hole openings 11 on whose inner walls the probes or capture molecules 20 can be arranged or immobilized. The pores 11 extend from a first surface or side 10A to a second surface or side 10B of the substrate 10 and are designed as through holes. If thus, for example, a DNA or RNA sample “hybridizes” with a nucleic acid probe containing a specific base sequence, the probe 20 binds (see at 22) to the nucleic acid target strand only if the sequences of the probe (capture molecule 20) and a target molecule 21 are completely or almost completely complementary.

It is then possible, according to the present invention, to detect the hybridization process by measuring the change in the light transmission property (properties) in the pore 11 in which the hybridization process took place and in which the hybridized probe 22 is arranged. To this end, light from a (white or monochromatic) light source 40 is coupled into the particular pores 11 via waveguides 30. In order to facilitate the coupling-in of individual light guides 30 into the particular pore 11, the corresponding ends 11A of the pores 11 may be designed in a conical or tapered shape. The light from the light source 40 enters the particular pore 11 via the front side of the waveguide 30, and its properties (such as intensity, diffraction properties, wavelength, phase, etc.) and the transmission properties of the pore 11 can change depending on whether or not the capture molecule(s) or probe(s) 20 arranged or immobilized therein has/have reacted with an appropriate analyte or target molecule 21. The light exiting from the pore 11 at the second surface or side 10B of the substrate 10 is measured and appropriately analyzed by a suitable detector 50, preferably a charged coupled device (CCD). In other words, light from pores 11H in which, for example, a hybridization has taken place supplies the corresponding area 50H of the CCD 50 with light which has different properties from the light which exits from pores 11NH in which no hybridization has taken place and which impinges on the corresponding areas 50NH of the CCD 50. If a phase shift of the transmitted light is to be studied, it is, however, necessary to study the light by means of an interferometer.

Attaching or coupling of, for example, oligonucleotides or DNA molecules to the inner wall surfaces of the pores 11 of the macroporous substrate 10 used according to the invention may be carried out according to the methods common in the prior art, for example by means of treating the porous substrate 10 with epoxysilanes and subsequent reaction of terminal epoxide groups with terminal primary amino groups or thiol groups of the oligonucleotides or DNA molecules used as capture molecules. In this connection, it is possible, for example, to prepare the oligonucleotides usable as capture molecules 20 in the present invention by using the synthesis strategy as described in Tet. Let. 22, 1981, pages 1859 to 1862. During the preparation process, the oligonucleotides may be derivatized with terminal amino groups either on the 5′ or 3′ terminals. Another possibility of attaching the capture molecules 20 to the inner wall surfaces of the pores 11 of, in particular, macroporous silicon 10 may be carried out by first treating the silicon substrate with a chlorine source such as Cl₂, SOCl₂, COCl₂ OR (COCl)₂, where appropriate by using a free radical initiator such as peroxides, azo compounds or Bu₃SnH, and subsequently reacting it with an appropriate nucleophilic compound, such as in particular with oligonucleotides or DNA molecules having terminal primary amino groups or thiol groups (see WO 00/33976).

The macroporous substrate 10 used usually has a pore diameter of from 500 nm to 100 μm, in particular 5 to 10 μm. The thickness of the macroporous substrate 10 is usually from 100 to 5,000 μm, preferably 200 to 500 μm. The thickness of the wall of the pores or through holes 11, i.e. the distance between two neighboring pores 11, is usually 1 to 2 μm. The pore density is usually in the range from 15 to 10⁸/cm², the pores 11 having an inner surface area of preferably 10 μm² to 3×10⁴ μm².

The macroporous substrate or the chip 10 is preferably made of macroporous silicon. The silicon may be doped, preferably n-doped, or undoped. A macroporous silicon of this kind may be prepared, for example, according to the method described in EP-A1-0 296 348. Silicon has the advantage of being impervious to light for the spectral range commonly used so that light which arrives at the first surface 10A of the macroporous substrate 10 constructed from silicon passes through the substrate 10 only through the pores 11 and not through the areas 12 arranged therebetween (i.e. the bulk silicon) and exits from the openings of the pores 11 on the second surface 10B of the substrate 10. In other words, a transmission peak develops close to the particular pore 11 and the correspondingly measured signal is essentially undisturbed by light passing through the bulk silicon 12.

The hole openings or pores 11 are preferably prepared electrolytically, with electrolytic etching being carried out in a hydrofluoric acid-containing electrolyte by applying a constant potential or a potential which changes over time, the layer consisting of silicon or the substrate 10 being connected as the positive electrode of an electrolytic cell. Holes 11 of this kind can be prepared, for example, as described in V. Lehmann, J. Electrochem. Soc. 140, 1993, pages 2836 et seq. Within the scope of the present invention, the macroporous substrate 10 provided may however also be, for example, other semiconducting substrates such as, for example, GaAs substrates or glass substrates coated with Si₃N₄.

Preferably, at least one capture molecule 20 per pore 11 is immobilized or bound location-specifically to the inner wall surfaces of at least some of the pores 11 (step (b)). In this connection, identical or different capture molecules 20 are applied to the substrate in the form of dots and essentially like a matrix, using an appropriate apparatus (not shown), an “arrayer”. Appropriate capillary forces distribute these liquid drops uniformly into one or more pores 11 in the macroporous substrate 10. This capillary distribution of the liquid has the advantage that air cannot enter the pores 11, since the throughput stops by itself when there is no longer any appropriate liquid. The side walls and inner wall surfaces of the pores 11 are generally occupied homogeneously with the appropriately used capture molecules or binding molecules 20. The capture molecules 20 are capable of undergoing a biochemical or chemical reaction such as, in particular, a sequence analysis by hybridization, an analysis of gene expression patterns by hybridization of mRNA or cDNA with gene-specific probes, an immunochemical analysis of protein mixtures, epitope mapping, an assay regarding receptor-ligand interactions and the profiling of cell populations, including binding of cell surface molecules to specific ligands or receptors. The capture molecules are preferably selected from the group consisting of DNA, proteins and ligands. Particular preference is given to using oligonucleotide probes as capture molecules.

For immobilizing, the macroporous substrate 10 may be derivatized, for example, with epoxysilane so that the capture molecules 20 such as, for example, oligonucleotide probes can be bound subsequently to the epoxysilane-derivatized substrate material via terminal amino groups.

Subsequently, an analyte 21 is contacted with the at lest one capture molecule 20 in at least one pore 11 (step (c)). In this connection, the analyte 21, i.e. the liquid to be studied, is usually pumped through the macropores 11. This may be achieved by building up a pressure gradient along the pores 11, usually in the range from 100 to 300 mbar, so that a pressure difference is generated between the first surface 10A and the second surface 10B of the macroporous substrate 10. For this purpose, the substrate 10 may be connected, for example, with an apparatus (not shown) which modifies dynamically and periodically the pressure in a closed volume located above the substrate 10 and tightly connected with said substrate.

The arrangement pattern of the pores 11 is usually designed, at least in some areas, according to a grid layout so that it can be scanned or sequentially addressed in the X-Y direction by automatic application and sampling devices such as, for example, samplers, pumps, suction lifters or the like, mouthpieces thereof, with in particular microvalves arranged in the same grid layout being controllable from the outside. Such microvalves themselves are known per se (cf. EP-A2-0 250 948). They are preferably arranged in the X-Y direction in the same array or in the same matrix as the pores 11 in the macroporous substrate 10 and therefore provide a possibility of a simple analysis for particular studies. The microvalves can be controlled and driven in a manner known per se.

DNA, RNA, PNA, saccharides, peptides, proteins, cell components, individual cells, multicellular organisms and cell assemblages may be used, for example, as analyte 21. The analyte 21 to be studied may be diluted, concentrated or metered. The dwell time can be controlled by appropriate closing and opening of the microvalves.

When the target molecules to be studied of the analyte 21 react with the capture molecules 20 immobilized on the inner wall surface of the pores 11 of the macroporous substrate 10 and bind to one other, the optical parameters or properties of the particular pore 11 in which the reaction occurs change. According to the present invention, the light transmission property of the at least one pore 11, which changes as a function of the occurrence of a binding reaction between the analyte 21 and the capture molecule 20 immobilized on the inner wall surface of the at least one pore 11, is measured or detected. Owing to the unique optical properties of porous silicon, as described in Applied Physics Letters, Volume 78, Number 5, 29 January 2001, the light transmission properties of the particular pore 11 change as a function of the occurrence of such a biochemical reaction. Depending on whether or not a reaction between the capture molecules 20 on the inner surface of the pore 11 and the target molecules to be studied of the analyte 21 has taken place in a pore 11 or a pore array, the property of the light which is coupled into the pore 11 or into the multiplicity of pores 11, for example by means of one or more light waveguides 30, is modified. On the basis of this, it is possible to detect according to the invention biochemical reactions such as, for example, the formation of DNA/DNA or RNA/DNA hybrids in the biochip. It is possible, within the scope of the present invention, also to measure, for example, the different absorption behavior of single-stranded and double-stranded DNA.

The change in the light transmission properties is measured (step (e)) by usually providing a support or bottom plate below the second surface 10B, which plate has a recording device 50 in the same arrangement for analysis on a microprocessor. In this connection, it is also possible to arrange a CCD array 50 or another appropriate detection unit, as is common in this specific field, tilted at an angle α to the macroporous substrate or the chip 10. Preferably, a CCD array is arranged below the second surface 10B. Such elements make it possible to store (preferably directly) the result of an assay or analysis, which can be specifically retrieved at any time, even if it is the result from individual pores 11 in the macroporous substrate 10 used according to the invention.

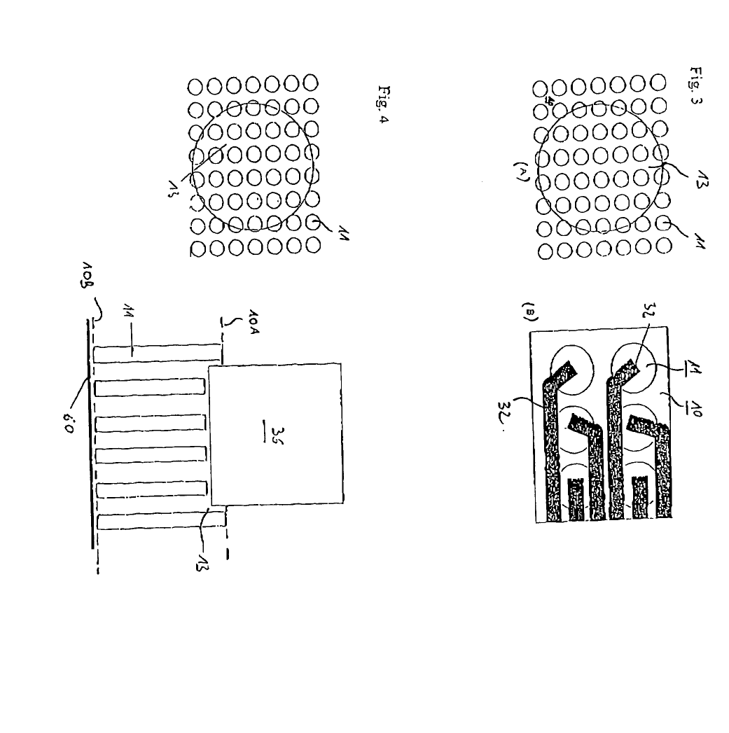

Preferably, planar light waveguides 32 (FIG. 3(B)) which are formed, for example, by waveguides whose front area through which, the light exits is beveled in a range from about 35° to about 55°, preferably by about 45°, so that light in the waveguide 32 is directed essentially parallel to the first surface 10A of the substrate 10 and is coupled into the particular pore 11 through the corresponding front areas of the waveguide 32, through which the light exits, preferably essentially perpendicular to the first surface 10A of the substrate 10, may be used for illuminating the first surface 10A with light (step (d)), preferably monochromatic light. It is furthermore possible, as an alternative or in addition, to use laser diodes which are in each case assigned either unambiguously to an individual pore 11 or to a group of neighboring pores 11 (FIG. 3(A)).

As shown in FIG. 3(B), it is likewise possible for the light transmitted through one or more pores 11 to be directed to the outside by one or more exit waveguides 34 (e.g. glass fibers). The diameter of the glass fiber 34 used may be identical to or in accordance with the dot size, i.e. several to several hundred pores 11, or may correspond to the diameter of a pore 11. In order to achieve a simpler connection and/or positioning of the exit waveguide 34, the corresponding surface 10B of the substrate 10 may be deepened, for example lithographically structured and etched by KOH, so that a rearward-protruding area 13 corresponding to the exit waveguide 34 is formed on the corresponding surface 10B. Such a rearward-protruding area can likewise be provided for coupling-in of light, i.e. on the first surface 10A of the substrate 10, in order to position a waveguide, for example a glass fiber (see, for example, FIG. 4).

Preferably, one or more glass fibers beveled by about 35° to 55°, preferably by about 45° (similar to the planar waveguide 32), for example, may be used for coupling out the light.

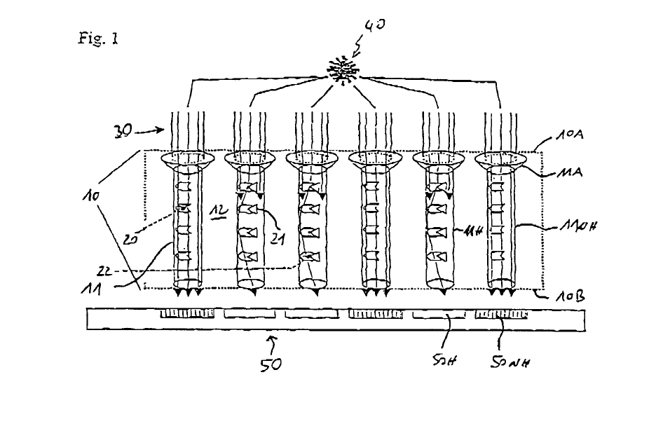

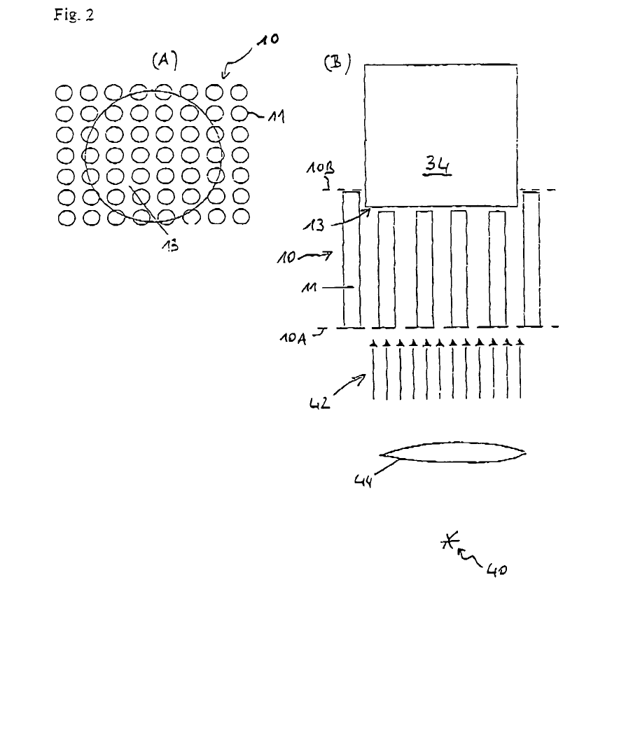

In one embodiment of the present invention, the light is coupled directly into at least one pore 11 through a light waveguide 30 and then, at the pore end on the second surface 10B of the macroporous substrate 10, directed to, for example, a CCD array 50 (FIG. 1). In another embodiment of the present invention, the light is coupled in and out through a light waveguide which covers a multiplicity of pores 11 of the macroporous substrate 10 (FIG. 2). An essentially homogeneous illumination of the pores 11 with light 42 on the first surface 10A can be generated from a light source 40 by an appropriate optical arrangement 44.

In another embodiment of the present invention (FIG. 4), the light coupled into the at least one pore 11 can be detected or measured in steps (d) and (e) after reflection on the rear side or the second surface 10B of the macroporous substrate 10. This method is particularly suitable for analyzing the phase information of the transmitted and reflected light (constructive and destructive interference, respectively). In this connection, the light supply device and the measuring device are arranged on the side of the first surface 10A and a reflecting agent 60 which reflects light transmitted through the pores 11 at least partially through the pores 11 into the measuring device is arranged on the side of the second surface 10B. In this connection, the transmitted light is reflected at the pore end or on the side of the second surface 10B of the substrate 10, again led through the corresponding pore(s) 11 and then, for example, coupled into a waveguide 36 (e.g. a glass fiber). The diameter of the glass fiber 36 may correspond almost to the dot size, and the glass fiber 36 may be fitted into a corresponding rearward-protruding area 13 of the first surface 10A. Thus the waveguide 36 can serve as a device for coupling in light, in order to couple light into the pores 11 of the substrate. The coupled-in light passes through the pores 11, i.e. is transmitted through these, its properties are, where appropriate, modified and it is reflected at or close to the second surface 10B of the substrate 10 by a reflecting device 60 (e.g. a mirror). The reflected light again passes through the corresponding pore(s) 11 and is coupled into the waveguide 34 at the first surface 10A and guided to a measuring or detecting device (not shown). Thus, a measurement of the transmitted light is also possible on the side of the first surface 10A of the substrate 10.

In order to achieve, when measuring the transmitted signal, a maximum difference between the pores 11H in which a reaction between the capture molecules 20 and the target molecules to be studied of the analyte 21 has taken place on the inner wall surface of the pore 11 and those pores 11NH in which no reaction or binding has taken place, it is possible, for example, to optimize the pore diameter, the pore length, the wavelength or wavelength range of the coupled-in light, the surface area of the pores 11 or the density of occupation by capture molecules 20 and the angle and/or distance at which the transmitted signal is measured.

Within the scope of the method of the invention it is possible to measure any changes in the transmission properties (in particular the diffraction properties) of the pores 11, in particular the change in the intensity of the transmitted signal, changes in the diffraction properties, wavelength changes or phase shifts. The change in intensity of the transmitted signal is preferably measured in step (e).

Coupling light into the pores 11 of the macroporous substrate 10 while measuring the change in the transmission properties as a function of the occurrence of a biochemical reaction results in the following advantages in principle:

an “optical” crosstalk from other dots during analysis is normally not possible, as a result of which the spatial resolution and thus the assignment between the particular dot and the detected signal is obtained automatically;

all pores of a dot contribute to the measured signal, resulting in a better signal-to-noise ratio;

the macroporous substrate used or the chip 10 used, in particular macroporous silicon, can be placed on a structured planar light waveguide 32 for readout, so that the chip 10 can be illuminated homogeneously from above and the transmitted signal can be directed via the light waveguide 32 to the side areas of the chip 10, thereby making possible a direct read-out in the variable inset plate (VIP). Such a variable inset plate (VIP) is described explicitly in the German patent application DE 100 27 104.9 and the European patent application 01 113 300.6. The patent applications mentioned whose disclosure content in this respect is intended to be part of the present application are hereby incorporated in their entirety by reference.

1. A method for detecting a biochemical reaction, the method comprising:
immobilizing a capture molecule on an inner wall of a pore selected from a multiplicity of pores extending between first and second opposed surfaces of a macroporous substrate;
contacting an analyte with the capture molecule;
directing light into the pore; and
measuring a light-transmission property of the pore, the light-transmission property being indicative of a binding reaction between the analyte and the capture molecule.

2. The method of claim 1, further comprising selecting a pore diameter of the pore to be between 500 nanometers and 100 micrometers.

3. The method of claim 1, further comprising selecting the macroporous substrate to be a macroporous silicon substrate.

4. The method of claim 1, further comprising selecting the macroporous substrate to have a thickness between 100 micrometers and 5000 micrometers.

5. The method of claim 1, further comprising distributing the multiplicity of pores so as to have a pore density in a range between 10⁵ pores per square centimeter and 10⁸ pores per square centimeter.

6. The method of claim 1, further comprising selecting the pore to have an inner surface area in a range between 10 square micrometers and 30,000 square micrometers.

7. The method of claim 1, further comprising distributing the multiplicity of pores in a grid.

8. The method of claim 7, further comprising scanning the grid with externally controlled microvalves arranged to correspond to the grid.

9. The method of claim 1, further comprising:
providing a support plate in optical communication with the second surface;
providing a recording device on the support plate; and
providing a microprocessor in communication with the recording device, the microprocessor being configured for analysis of data collected by the recording device.

10. The method of claim 9, wherein providing a recording device comprises providing a CCD array in optical communication with the second surface.

11. The method of claim 10, further comprising tilting the CCD array relative to the second surface.

12. The method of claim 1, wherein directing light into the pore comprises providing a waveguide for coupling light directly into the pore.

13. The method of claim 12, wherein providing a waveguide comprises selecting a waveguide that covers a multiplicity of pores.

14. The method of claim 1, wherein measuring a light-transmission property comprises detecting light that has been reflected back into the pore.

15. The method of claim 1, wherein directing light into the pore comprises providing a planar light waveguide in optical communication with the pore.

16. The method of claim 1, wherein directing light into the pore comprises providing a laser diode in optical communication with the pore.

17. The method of claim 1, wherein directing light into the pore comprises providing a glass fiber in optical communication with the pore.

18. The method of claim 17, wherein providing a glass fiber comprises providing a fiber having a beveled end in optical communication with the pore.

19. The method of claim 18, wherein providing a glass fiber having a beveled end comprises selecting the bevel angle to be in the range between 35 degrees and 55 degrees relative to the first surface.

20. The method of claim 19, wherein selecting the bevel angle comprises selecting the angle to be approximately 45 degrees.

21. The method of claim 1, further comprising selecting the capture molecule from the group consisting of DNA, proteins, and ligands.

22. The method of claim 21, further comprising selecting the capture molecule to be an oligonucleotide probe.

23. The method of claim 22, further comprising:
derivatizing the substrate with epoxysilane, and
binding the oligonucleotide probe with a terminal group selected from the group consisting of an amino group and a thiol group.

24. The method of claim 1, further comprising selecting the analyte from the group consisting of DNA, RNA, PNA, saccharides, peptides, proteins, cell components, individual cells, multicellular organisms, and cell assemblages.

25. An apparatus for detecting a biochemical reaction, the apparatus comprising:
a macroporous substrate having
a first surface,
a second surface opposed to the first surface, and
a multiplicity of pores extending between the first and second surfaces;
a light supply device for supplying light to a pore selected from the multiplicity of pores;
a capture molecule immobilized at an inner wall of the pore; and
a measuring device for measuring a light-transmission property of the pore, the light-transmission property being indicative of an occurrence of a binding reaction between an analyte and the capture molecule.

26. The apparatus of claim 25, wherein the macroporous substrate comprises a macroporous silicon substrate.

27. The apparatus of claim 25, wherein the pores are disposed in a grid.

28. The apparatus of claim 22, further comprising automatic application and sampling devices for automatically scanning the pores.

29. The apparatus of claim 28, wherein the automatic application and sampling devices comprise externally controllable microvalves disposed on a grid.

30. The apparatus of claim 25, wherein the measuring device comprises a CCD array in optical communication with the second surface.

31. The apparatus of claim 30, wherein the CCD array is tilted relative to the second surface.

32. The apparatus of claim 25, wherein the light supply device comprises a waveguide disposed to couple light directly into the pore.

33. The apparatus of claim 25, wherein the light supply device comprises a waveguide arranged to couple light into a multiplicity of pores.

34. The apparatus of claim 32, wherein the waveguide comprises a planar waveguide disposed on the first surface.

35. The apparatus of claim 25, wherein the light supply device comprises a laser diode disposed to couple light into the pore.

36. The apparatus of claim 25, wherein the light supply device comprises a glass fiber having a beveled end, the beveled end being disposed to couple light into the pore.

37. The apparatus of claim 36, wherein the beveled end is beveled at an angle in the range between 35 degrees and 55 degrees.

38. The apparatus of claim 37, wherein the beveled end is beveled at an angle of approximately 45 degrees.

39. The apparatus of claim 25, wherein the capture molecule is selected from the group consisting of DNA, proteins, and ligands.

40. The apparatus of claim 39, wherein the capture molecule comprises an oligonucleotide probe.

41. The apparatus of claim 25,
further comprising a reflecting agent disposed in optical communication with the second surface, and
wherein the measuring device is disposed to detect light reflected back from the reflecting agent.
